(19) United States
(12) Patent Application Publication
(10) Pub. No.: US 2021/0061500 A1
(43) Pub. Date: Mar. 4, 2021

(54) SPACESUIT WITH LIQUID COOLING VENTILATION GARMENT, SOFT EXOSKELETON, AND BIOSENSORS

(71) Applicant: Lockheed Martin Corporation, Bethesda, MD (US)

(72) Inventors: Gavin A. Barnes, St. Cloud, FL (US); Vanessa A. Williams, Littleton, CO (US); Michael J. Aselin, Houston, TX (US)

(21) Appl. No.: 17/011,459

(22) Filed: Sep. 3, 2020

Related U.S. Application Data
(60) Provisional application No. 62/895,666, filed on Sep. 4, 2019.

(51) Int. Cl. B64G 6/00; A41D 13/005; A62B 17/00; A61F 7/00; A61H 3/00

(52) U.S. Cl. CPC B64G 6/00; A41D 13/005; A62B 17/005; A62B 17/008; A61F 7/00; A61B 5/0531; A61H 2003/007; A61H 2230/605; A61F 2007/0093; A61F 2007/0086; A61H 3/00

(57) ABSTRACT

Disclosed herein is a spacesuit including a liquid cooling ventilation garment, a soft exoskeleton, at least one biometric sensor, and an electronic controller in electronic communication with the liquid cooling ventilation garment, the soft exoskeleton, and the at least one biometric sensor. The electronic controller is configured to operate the soft exoskeleton based on electromyography data of the at least one biometric sensor to produce a desired change in orientation of the soft exoskeleton and corresponding user. The electronic controller is further configured to operate the liquid cooling ventilation garment based on temperature data of the at least one biometric sensor to maintain a user temperature within a predetermined user temperature range. In certain embodiments, the liquid cooling ventilation garment is in thermal communication with the soft exoskeleton and used for thermal management thereof. Such configurations reduce the physical and cognitive loading of the astronaut.

### RELATED APPLICATIONS

This application claims the benefit of U.S. Provisional Application No. 62/895,666, filed on Sep. 4, 2019, entitled “SPACESUIT WITH LIQUID COOLING VENTILATION GARMENT, SOFT EXOSKELETON, AND BIOSENSORS,” the disclosure of which is hereby incorporated herein by reference in its entirety.

### BACKGROUND

Spacesuits are required to protect humans from the extreme environment of space, particularly for extravehicular activity (EVA). Due to the nature of the space environment, spacesuits are often stiff and bulky. For example, spacesuits generally have a high material stiffness from multiple layers of protection (e.g., thermal layer, pressure bladder, comfort layer, etc.). Further, air pressure from artificial internal atmosphere within the spacesuit may stiffen the spacesuit (e.g., drive the joints to a fully extended position). Due to this increased physical loading on the astronaut, movement by astronauts in spacesuits can require considerable effort, which may lead to astronaut fatigue, reduced operation time, reduced manual dexterity, and/or suit damage from alternative locomotion methods, etc.

Some spacesuits include liquid cooling ventilation garments (LCVG) to maintain a comfortable core body temperature during EVA. Often, LCVGs are close-fitting undergarments with flexible tubing to circulate cool water to remove excess body heat. An astronaut may manually control such garments to control their own temperature. However, an optimal temperature may vary depending on the environment and nature of the tasks being performed, among other factors. Accordingly, such manual operation increases the cognitive loading on the astronaut.

### SUMMARY

Disclosed herein is a spacesuit including a liquid cooling ventilation garment, a soft exoskeleton, at least one biometric sensor, and an electronic controller in electronic communication with the liquid cooling ventilation garment, the soft exoskeleton, and the at least one biometric sensor. The electronic controller is configured to operate the soft exoskeleton based on electromyography data of the at least one biometric sensor to produce a desired change in orientation of the soft exoskeleton and corresponding user. The electronic controller is further configured to operate the liquid cooling ventilation garment based on the temperature data to maintain a user temperature within a predetermined user temperature range. In certain embodiments, the liquid cooling ventilation garment is in thermal communication with the soft exoskeleton and used for thermal management thereof. Such configurations reduce the physical and cognitive loading of the astronaut.

In one embodiment, a spacesuit includes a liquid cooling ventilation garment, a soft exoskeleton, at least one biometric sensor, and an electronic controller in electronic communication with the liquid cooling ventilation garment, the soft exoskeleton, and the at least one biometric sensor. The at least one biometric sensor is configured to measure temperature to generate temperature data, and measure electric potential to generate electromyography data. The electronic controller is configured to operate the soft exoskeleton based on the electromyography data to produce a desired change in orientation of the soft exoskeleton, and operate the liquid cooling ventilation garment based on the temperature data to maintain a user temperature within a predetermined user temperature range.

In another embodiment, a method of operating a spacesuit includes receiving, by an electronic controller, sensor data from at least one biometric sensor of a spacesuit. The sensor data includes temperature data and electromyography data. The method further includes operating, by the electronic controller, a soft exoskeleton of the spacesuit based on the electromyography data to produce a desired change in orientation of the soft exoskeleton. The method further includes operating, by the electronic controller, a liquid cooling ventilation garment of the spacesuit based on at least one of the electromyography data or the temperature data to maintain a user temperature within a predetermined user temperature range.

In another embodiment, a spacesuit includes a liquid cooling ventilation garment including at least one channel and at least one pump configured to pump a cooling liquid through the at least one channel. The spacesuit further includes a soft exoskeleton including at least one drive cable and at least one actuator configured to operate the at least one drive cable. The soft exoskeleton is in thermal communication with the liquid cooling ventilation garment. The spacesuit further includes at least one biometric sensor configured to measure temperature to generate temperature data, and measure electric potential to generate unconditioned electromyography data. The spacesuit further includes an electronic controller in electronic communication with the liquid cooling ventilation garment, the soft exoskeleton, and the at least one biometric sensor. The electronic controller is configured to receive sensor data from the at least one biometric sensor. The sensor data includes the temperature data and the unconditioned electromyography data. The electronic controller is further configured to condition the unconditioned electromyography data based on the temperature data to generate conditioned electromyography data. The electronic controller is further configured to process the conditioned electromyography data to determine a desired change in orientation of the soft exoskeleton. The electronic controller is further configured to operate the at least one actuator to drive the at least one drive cable to move the soft exoskeleton according to the desired change in orientation of the soft exoskeleton. The electronic controller is further configured to process the temperature data to determine an operational change of the liquid cooling ventilation garment based on at least one of a current temperature of the user, a current temperature of the soft exoskeleton, an anticipated temperature increase of the user within a predetermined time period based on movement of the soft exoskeleton according to the desired change in orientation, or an anticipated temperature increase of the soft exoskeleton within a predetermined time period based on movement of the soft exoskeleton according to the desired change in orientation. The electronic controller is further configured to operate the at least one pump to modify pumping of the cooling liquid through the at least one channel to maintain at least one of a user temperature within a predetermined user temperature range, or an exoskeleton temperature within a predetermined exoskeleton temperature range.

Those skilled in the art will appreciate the scope of the disclosure and realize additional aspects thereof after reading the following detailed description of the embodiments in association with the accompanying drawing figures.

### BRIEF DESCRIPTION OF THE DRAWINGS

The accompanying drawing figures incorporated in and forming a part of this specification illustrate several aspects of the disclosure and, together with the description, serve to explain the principles of the disclosure.

FIG. 1 is a diagram illustrating a spacesuit including a liquid cooling ventilation garment, a soft exoskeleton, and biosensors.

FIG. 2 is a diagram illustrating the liquid cooling and ventilation garment of the spacesuit of FIG. 1.

FIG. 3 is a diagram illustrating the soft exoskeleton of the spacesuit of FIG. 1.

FIG. 4 is a diagram illustrating the spacesuit of FIG. 1 mounted to a user.

FIG. 5 is a flowchart illustrating operation of the spacesuit of FIG. 1.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

The use herein of ordinals in conjunction with an element is solely for distinguishing what might otherwise be similar or identical labels, such as “first format” and “second format,” and does not imply a priority, a type, an importance, or other attribute, unless otherwise stated herein. The term “about” used herein in conjunction with a numeric value means any value that is within a range of ten percent greater than or ten percent less than the numeric value.

As used herein and in the claims, the articles “a” and “an” in reference to an element refers to “one or more” of the element unless otherwise explicitly specified. The word “or” as used herein and in the claims is inclusive unless contextually impossible. As an example, the recitation of A or B means A, or B, or both A and B.

Any flowcharts discussed herein are necessarily discussed in some sequence for purposes of illustration, but unless otherwise explicitly indicated, the embodiments are not limited to any particular sequence of steps. The use herein of ordinals in conjunction with an element is solely for distinguishing what might otherwise be similar or identical labels, such as “first format” and “second format,” and does not imply a priority, a type, an importance, or other attribute, unless otherwise stated herein. The term “about” used herein in conjunction with a numeric value means any value that is within a range of ten percent greater than or ten percent less than the numeric value.

As used herein, the term “electromyography” or “EMG” refers to the detection and recording of the electrical activity of muscle tissue resulting from electrochemical reactions in the muscle tissue.

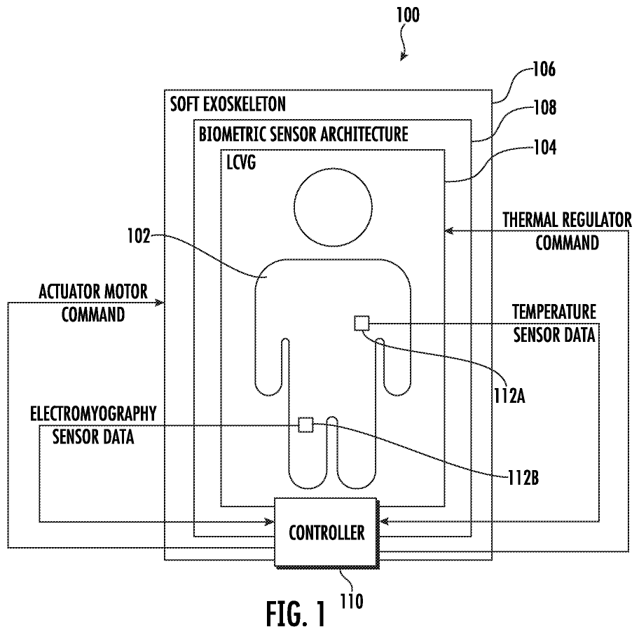

FIG. 1 is a diagram illustrating a spacesuit 100 of a user 102 (i.e., astronaut) including a liquid cooling ventilation garment (LCVG) 104, a soft exoskeleton 106, and biometric sensor architecture 108. The spacesuit 100 further includes an electronic controller 110 in electronic communication with the LCVG 104, the soft exoskeleton 106, and the biometric sensor architecture 108 includes at least one biometric sensor (e.g., a first biometric sensor 112A and a second biometric sensor 112B).

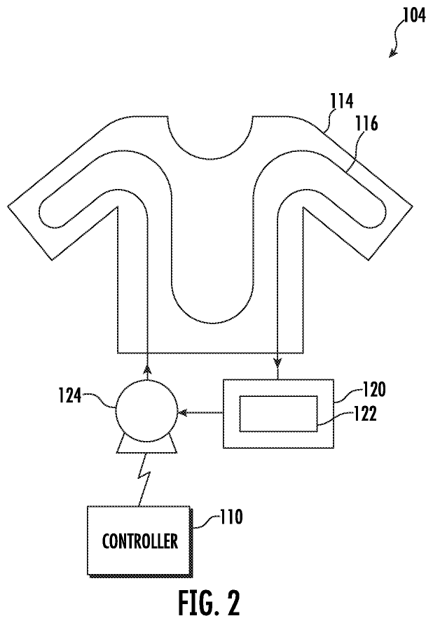

Referring to FIG. 2, the LCVG 104 includes a fabric material 114 forming a clothing garment (e.g., pants, shirt, glove, etc.) and at least one channel 116 attached to and/or formed within the fabric material 114. In certain embodiments, the fabric material 114 includes nylon (e.g., nylon tricot), spandex, and/or other compliant materials. In certain embodiments, the at least one channel 116 includes tubing (e.g., plastic tubing). Further, in certain embodiments, the LCVG 104 includes one or more ventilation ducts to draw moist air from the user's extremities to keep the user dry.

For circulation of a cooling liquid through the at least one channel 116, the LCVG 104 further includes a reservoir 120, a cooling source 122 (e.g., ice, ice pack, refrigerator, cooling device, etc.), and a pump 124. The reservoir 120 contains a cooling liquid (e.g., water). In certain embodiments, the cooling source 122 includes an ice pack positioned within the reservoir 120. In other embodiments, the cooling source 122 is external to the reservoir 120.

The at least one pump 124 is configured to pump the cooling liquid through the at least one channel 116. In this way, cooling liquid is pumped from the reservoir 120 through the at least one channel 116 and across the fabric material 114 to cool the user 102. The temperature of the cooling liquid rises as the cooling liquid moves through the at least one channel 116, until returning to the reservoir 120, where the cooling liquid is cooled by the cooling source 122. Accordingly, the cooling liquid may be reused to cool a user 102.

The LCVG 104 is in thermal communication with the user 102, the soft exoskeleton 106, and/or the at least one biometric sensor 112A, 1128 (may be referred to generally as biometric sensor 112). In particular, for example, the pump 124 and/or cooling source 122 is in electronic communication with the controller 110. Accordingly, the LCVG 104 can be used for thermal management and temperature control of the user 102, the soft exoskeleton 106, and/or the at least one biometric sensor 112. In certain embodiments, the LCVG 104 uses plate-to-plate cooling to cool electronics of the soft exoskeleton 106.

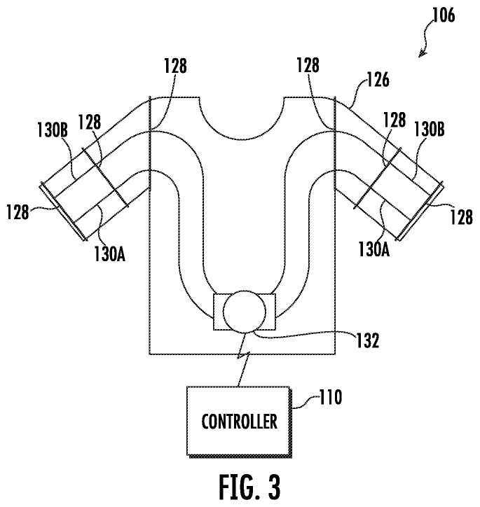

Referring to FIG. 3, in certain embodiments, the soft exoskeleton 106 (may also be referred to as a soft exosuit) is devoid of rigid elements. In this way, the soft exoskeleton 106 conforms to a human body such that the user's joints are unconstrained by external rigid structures and is generally lighter than other systems that use rigid structures. In certain embodiments, the soft exoskeleton 106 includes a fabric material 126 forming a clothing garment (e.g., pants, shirt, glove, etc.) and at least one strap 128. In certain embodiments, the fabric material 126 includes fabric, rubber, wire, and/or other compliant materials. In certain embodiments, the at least one strap 128 is made of a more resilient and/or tougher material than the fabric material 126.

In certain embodiments, the soft exoskeleton 106 includes at least one drive cable 130 and at least one actuator 132 configured to operate the at least one drive cable 130A, 1308 (may be referred to generally as drive cable 130). The at least one drive cable 130 is attached to one or more straps 128. The drive cables 130 may be configured, such that the drive cables 130 only pull the straps 128. For example, a first drive cable 130A may be positioned under the elbow so that pulling the first drive cable 130A extends a user's arm outward. A second drive cable 1308 may be positioned over the elbow so that pulling the second drive cable 1308 bends the arm inward.

In certain embodiments, the actuator 132 includes at least one of a cable actuator or an air actuator (e.g., pneumatic air actuator, pneumatic artificial muscle, etc.). Operation of the actuator 132 pulls on one or more drive cables 130, which are attached to one or more straps 128, which are fixed to a user 102. Accordingly, the soft exoskeleton 106 provides assistance or resistance to a user 102 by pulling with or against a user's movements.

The soft exoskeleton 106 is a low-profile, ultra-lightweight actuation solution which can provide the user 102 wearing the soft exoskeleton 106 with assistance or resistance, depending on the intended application. In certain embodiments, it assists the user 102 when mitigating the constant extension torque induced by both the material stiffness of a concurrently worn spacesuit and when working against the air pressure of the artificial atmosphere when deforming the spacesuit. The soft exoskeleton 106 provides additional force to the user 102's movements to hold the rigid spacesuit in place so the user 102 does not have to.

The soft exoskeleton 106 resists the user 102 when worn while exercising, such that the soft exoskeleton 106 impedes the user 102's ability to extend or flex their limbs. It does this by providing varying levels of force resistance during flexion and extension activities. The soft exoskeleton 106 is in thermal communication with the LCVG 104. Further, in certain embodiments, the soft exoskeleton 106 can act as a chair for the user 102, such as to support the user while landing. Such a configuration may reduce hardware needs and burdens on the user's joints (e.g., knees), as current configurations provide complicated harnessing that is limited to a one-time use.

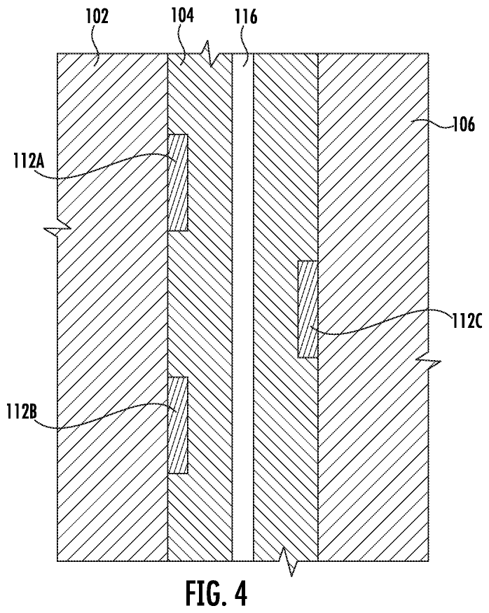

Referring to FIGS. 1 and 4, the at least one biometric sensor 112 includes, for example, a first biometric sensor 112A (e.g., temperature sensor) configured to measure temperature to generate temperature data, and a second biometric sensor 112B (e.g., electromyography sensor) configured to measure electric potential to generate unconditioned electromyography (EMG) data. In certain embodiments, the at least one biometric sensor 112 is further configured to measure skin impedance to produce perspiration data. In certain embodiments, at least some of the biometric sensors 112 are configured to be coupled to a user 102, such as by electrodes with adhesives.

In certain embodiments, additional biometric sensors may be used. For example, in certain embodiments, the first and second biometric sensors 112A, 1128 are positioned on or towards a first side of the LCVG 104 and in contact with a user 102, and a third biometric sensor 112C (e.g., temperature sensor) is positioned on or towards a second side of the LCVG 104 to measure temperature to generate temperature data of the soft exoskeleton 106. Of course, more biometric sensors 112 may be used, and more of each type of biometric sensor 112 may be used.

The first biometric sensor 112A provides feedback as to the temperature of the user 102, which can be used to drive a thermal regulator function so the user 102 stays in a desired thermal state. Doing so prevents adding an additional cognitive load to the user 102 created by the user 102 manually controlling their own cooling system.

The second biometric sensor 1128 provides feedback as to the EMG of the user 102, which can be used to drive a mechanical regulator function so the soft exoskeleton 106 is more responsive. In particular, EMG measures electrical activity of a muscle during rest, slight contraction and forceful contraction. As muscle tissue does not normally produce electrical signals during rest, measurement of EMG can be used to anticipate and/or follow a user's movement and affect the soft exoskeleton 106 to move with and assist the user's movement.

EMG sensors 1128 may be used generate actuator commands at a speed that causes movement of a drive cable 130 within milliseconds of a contraction of a user's muscles. In particular, in response to a contraction of a muscle of a human user, an EMG sensor 1128 generates EMG sensor data that quantifies the muscle contraction. A controller 110 receives the EMG sensor data, determines an actuator command based on the EMG sensor data, and communicates the actuator command to the actuator 132.

One advantage of this arrangement is that the soft exoskeleton 106 can be instructed to move in response to the muscle contraction of the user 102 before the muscle causes the user's joint to actually move. Since the delay between the electrochemical reaction, which causes the muscle contraction, and the actual movement of the human user's joint is greater than the time required for the EMG sensor 1128 and the controller 110 to generate and utilize the EMG sensor data to cause the actuator to move, the soft exoskeleton 106 can accurately mimic the actual movements of the user 102 (e.g., without the need for conventional reactive or predictive systems). When EMG is used with skeletal muscle tissue, i.e., muscles operatively connected to one or more skeletal bones (or other types of body segments), EMG can detect contraction of the muscle tissue before the muscle tissue has contracted with sufficient force to actually move the joint.

EMG sensors provide assistance to more dynamic movement profiles than force or kinematic-based controllers. In certain embodiments, the second biometric sensor 1128 includes a sensor with a foam path and gel coating and an electrode slightly raised from the skin of the user 102 and/or an antenna in direct contact with the skin of the user 102. In certain embodiments, the second biometric sensor 1128 is used to optimize user exertion by tracking EMG data during resistance activities for workouts.

The third biometric sensor 112C provides feedback as to the temperature of the soft exoskeleton 106, which can be used to drive a thermal regulator function so the soft exoskeleton 106 (or a portion thereof) stays in a desired thermal state.

Referring to FIG. 1, in certain embodiments, the electronic controller 110 is in electronic communication with the LCVG 104, the soft exoskeleton 106, and/or the at least one biometric sensor 108. The electronic controller 110 is configured to receive sensor data from the at least one biometric sensor 108. In certain embodiments, the sensor data includes the temperature data and/or the unconditioned electromyography data.

The electronic controller 110 is configured to operate the soft exoskeleton 106 based on the electromyography data to produce a desired change in orientation of the soft exoskeleton 106 (and corresponding user 102). In particular, the electronic controller 110 is further configured to condition the unconditioned electromyography data based on the temperature data to generate conditioned electromyography data. In particular, temperature may affect sensor readings. For example, if the second biometric sensor 1128 is hot, then the unconditioned EMG data may not be completely accurate. The electronic controller 110 processes the unconditioned electromyography data based on the temperature data to produce a more accurate EMG value. The electronic controller 110 is further configured to process the conditioned electromyography data to determine a desired change in orientation of the soft exoskeleton 106 (and corresponding user 102). The electronic controller 110 is further configured to operate the at least one actuator 132 (see FIG. 3), to drive the at least one drive cable 130 to move the soft exoskeleton 106 according to the desired change in orientation of the soft exoskeleton 106 (and corresponding user 102). In this way, compared to the unconditioned electromyography data, the conditioned electromyography data more accurately reflects the intended motion of the user 102.

The electronic controller 110 is further configured to operate the LCVG 104 based on the temperature data to maintain a user temperature within a predetermined user temperature range. In particular, the electronic controller 110 is further configured to process the temperature data to determine an operational change of the LCVG 104 based on at least one of a current temperature of the user 102, a current temperature of the soft exoskeleton 106, an anticipated temperature increase of the user 102 within a predetermined time period based on movement of the soft exoskeleton 106 according to the desired change in orientation, and/or an anticipated temperature increase of the soft exoskeleton 106 within a predetermined time period based on movement of the soft exoskeleton 106 according to the desired change in orientation. In other words, in certain embodiments, the electronic controller 110 is able to anticipate the temperature change of the user 102 and/or soft exoskeleton 106 based on the sensor data of the at least one biometric sensor 108. The electronic controller 110 is further configured to operate the at least one pump to modify pumping of the cooling liquid through the at least one channel 116 to maintain at least one of a user temperature within a predetermined user temperature range, and/or an exoskeleton temperature within a predetermined exoskeleton temperature range. In certain embodiments, the electronic controller 110 is further configured to operate the LCVG 104 based on the perspiration data.

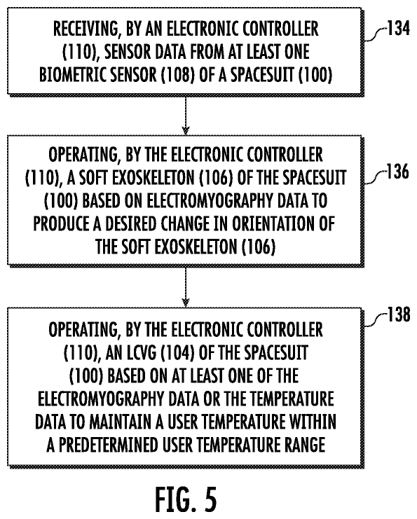

FIG. 5 is a flowchart illustrating operation of the spacesuit 100 of FIG. 1. In step 134, the method includes receiving, by the electronic controller 110, sensor data from at least one biometric sensor 108 of the spacesuit 100, the sensor data comprising temperature data and electromyography data.

In step 136, the method includes operating, by the electronic controller 110, the soft exoskeleton 106 of the spacesuit 100 based on electromyography data to produce a desired change in orientation of the soft exoskeleton 106 (and corresponding user 102).

In step 138, the method includes operating, by the electronic controller 110, the LCVG 104 of the spacesuit 100 based on at least one of the electromyography data or the temperature data to maintain a user temperature within a predetermined user temperature range.

In certain embodiments, the method further includes operating, by the electronic controller 110, the LCVG 104 to maintain an exoskeleton temperature within a predetermined exoskeleton temperature range, where the soft exoskeleton 106 is in thermal communication with the LCVG 104.

In certain embodiments, the method includes conditioning unconditioned electromyography data from the at least one biometric sensor 108 based on the temperature data to generate conditioned electromyography data. The method further includes processing the conditioned electromyography data to determine the desired change in orientation of the soft exoskeleton 106 (and corresponding user 102).

In certain embodiments, the method further includes operating, by the electronic controller 110, the LCVG 104 to maintain a user temperature within a predetermined user temperature range, and an exoskeleton temperature within a predetermined exoskeleton temperature range.

In certain embodiments, the method further includes processing, by the electronic controller 110, the temperature data to determine an operational change of the LCVG 104 based on at least one of a current temperature of the user 102, a current temperature of the soft exoskeleton 106, an anticipated temperature increase of the user 102 within a predetermined time period based on movement of the soft exoskeleton 106 according to the desired change in orientation, and/or an anticipated temperature increase of the soft exoskeleton 106 within a predetermined time period based on movement of the soft exoskeleton 106 according to the desired change in orientation.

Those skilled in the art will recognize improvements and modifications to the preferred embodiments of the disclosure. All such improvements and modifications are considered within the scope of the concepts disclosed herein and the claims that follow.

What is claimed is:

1. A spacesuit, comprising:
a liquid cooling ventilation garment;
a soft exoskeleton;
at least one biometric sensor configured to:
measure temperature to generate temperature data; and
measure electric potential to generate electromyography data; and
an electronic controller in electronic communication with the liquid cooling ventilation garment, the soft exoskeleton, and the at least one biometric sensor, the electronic controller configured to:
operate the soft exoskeleton based on the electromyography data to produce a desired change in orientation of the soft exoskeleton; and
operate the liquid cooling ventilation garment based on the temperature data to maintain a user temperature within a predetermined user temperature range.

2. The spacesuit of claim 1, wherein the liquid cooling ventilation garment comprises at least one channel and at least one pump configured to pump a cooling liquid through the at least one channel.

3. The spacesuit of claim 1,
wherein the liquid cooling ventilation garment is in thermal communication with the soft exoskeleton; and
wherein the electronic controller is further configured to maintain an exoskeleton temperature within a predetermined exoskeleton temperature range.

4. The spacesuit of claim 1, wherein the soft exoskeleton includes at least one drive cable and at least one actuator configured to operate the at least one drive cable.

5. The spacesuit of claim 4, wherein the at least one actuator comprises at least one of a cable actuator or an air actuator.

6. The spacesuit of claim 1,
wherein the at least one biometric sensor is further configured to measure skin impedance to produce perspiration data; and
wherein the electronic controller is further configured to operate the liquid cooling ventilation garment based on the perspiration data.

7. The spacesuit of claim 1,
wherein the at least one biometric sensor is further configured to measure electric potential to generate unconditioned electromyography data; and
wherein the electronic controller is further configured to:
condition the unconditioned electromyography data based on the temperature data to generate conditioned electromyography data; and
process the conditioned electromyography data to determine the desired change in orientation of the soft exoskeleton.

8. The spacesuit of claim 1, wherein the at least one biometric sensor is further configured to measure a current temperature of the user and a current temperature of the soft exoskeleton.

9. The spacesuit of claim 8, wherein the electronic controller is configured to operate the liquid cooling ventilation garment to maintain:
an exoskeleton temperature within a predetermined exoskeleton temperature range.

10. The spacesuit of claim 1, wherein the electronic controller is further configured to process the temperature data to determine an operational change of the liquid cooling ventilation garment based on at least one of:
a current temperature of the user;
a current temperature of the soft exoskeleton;
an anticipated temperature increase of the user within a predetermined time period based on movement of the soft exoskeleton according to the desired change in orientation; or
an anticipated temperature increase of the soft exoskeleton within a predetermined time period based on movement of the soft exoskeleton according to the desired change in orientation.

11. The spacesuit of claim 1, wherein the electronic controller is further configured to process the temperature data to determine an operational change of the liquid cooling ventilation garment based on:
a current temperature of the user;
a current temperature of the soft exoskeleton;
an anticipated temperature increase of the user within a predetermined time period based on movement of the soft exoskeleton according to the desired change in orientation; and
an anticipated temperature increase of the soft exoskeleton within a predetermined time period based on movement of the soft exoskeleton according to the desired change in orientation.

12. A method of operating a spacesuit, comprising:
receiving, by an electronic controller, sensor data from at least one biometric sensor of the spacesuit, the sensor data comprising temperature data and electromyography data;
operating, by the electronic controller, a soft exoskeleton of the spacesuit based on the electromyography data to produce a desired change in orientation of the soft exoskeleton; and
operating, by the electronic controller, a liquid cooling ventilation garment of the spacesuit based on at least one of the electromyography data or the temperature data to maintain a user temperature within a predetermined user temperature range.

13. The method of claim 12, wherein the liquid cooling ventilation garment comprises at least one channel and at least one pump configured to pump a cooling liquid through the at least one channel.

14. The method of claim 12, further comprising operating, by the electronic controller, the liquid cooling ventilation garment to maintain an exoskeleton temperature within a predetermined exoskeleton temperature range, the soft exoskeleton in thermal communication with the liquid cooling ventilation garment.

15. The method of claim 12, wherein the soft exoskeleton includes at least one drive cable and at least one actuator configured to operate the at least one drive cable.

16. The method of claim 12, further comprising:
conditioning unconditioned electromyography data from the at least one biometric sensor based on the temperature data to generate conditioned electromyography data; and
processing the conditioned electromyography data to determine the desired change in orientation of the soft exoskeleton.

17. The method of claim 12, wherein the temperature data comprises a current temperature of the user and a current temperature of the soft exoskeleton.

18. The method of claim 17, further comprising operating, by the electronic controller, the liquid cooling ventilation garment to maintain:
an exoskeleton temperature within a predetermined exoskeleton temperature range.

19. The method of claim 12, further comprising processing, by the electronic controller, the temperature data to determine an operational change of the liquid cooling ventilation garment based on at least one of:
a current temperature of the user;
a current temperature of the soft exoskeleton;
an anticipated temperature increase of the user within a predetermined time period based on movement of the soft exoskeleton according to the desired change in orientation; and
an anticipated temperature increase of the soft exoskeleton within the predetermined time period based on the movement of the soft exoskeleton according to the desired change in orientation.

20. A spacesuit, comprising:
a liquid cooling ventilation garment comprising at least one channel and at least one pump configured to pump a cooling liquid through the at least one channel;
a soft exoskeleton comprising at least one drive cable and at least one actuator configured to operate the at least one drive cable, the soft exoskeleton in thermal communication with the liquid cooling ventilation garment;
at least one biometric sensor configured to:
measure temperature to generate temperature data; and
measure electric potential to generate unconditioned electromyography data; and
an electronic controller in electronic communication with the liquid cooling ventilation garment, the soft exoskeleton, and the at least one biometric sensor, the electronic controller configured to:
receive sensor data from the at least one biometric sensor, the sensor data comprising the temperature data and the unconditioned electromyography data;
condition the unconditioned electromyography data based on the temperature data to generate conditioned electromyography data;
process the conditioned electromyography data to determine a desired change in orientation of the soft exoskeleton;
operate the at least one actuator to drive the at least one drive cable to move the soft exoskeleton according to the desired change in orientation of the soft exoskeleton; and
process the temperature data to determine an operational change of the liquid cooling ventilation garment based on at least one of:
a current temperature of the user;
a current temperature of the soft exoskeleton;
an anticipated temperature increase of the user within a predetermined time period based on movement of the soft exoskeleton according to the desired change in orientation; or
an anticipated temperature increase of the soft exoskeleton within a predetermined time period based on the movement of the soft exoskeleton according to the desired change in orientation; and
operate the at least one pump to modify pumping of the cooling liquid through the at least one channel to maintain at least one of:
a user temperature within a predetermined user temperature range; or
an exoskeleton temperature within a predetermined exoskeleton temperature range.
